(19) United States
(12) Patent Application Publication
(10) Pub. No.: US 2008/0124807 A1
(43) Pub. Date: May 29, 2008

(54) Protein tagging reagents

(76) Inventors: Christian Schoneich, Lawrence, KS (US); John F. Stobaugh, Lawrence, KS (US); Xiaobao Li, Lawrence, KS (US); Justin Pennington, Bound Brook, NJ (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 11/906,528

(22) Filed: Oct. 2, 2007

Related U.S. Application Data
(60) Provisional application No. 60/848,862, filed on Oct. 2, 2006.

(51) Int. Cl. G01N 33/00; G01N 37/00; C07D 487/02; C07K 7/00

(52) U.S. Cl. 436/56; 530/300; 436/86; 436/63; 548/453

(57) ABSTRACT

Benzylamine-like reagents for the affinity enrichment and relative quantification of post-translational hydroxylation and nitration of tyrosine and tryptophan in proteins are provided.

### CROSS-REFERENCE TO RELATED APPLICATIONS

This application is based on and claims priority to U.S. Provisional Application Ser. No. 60/848,862, filed on Oct. 2, 2006 which is incorporated herein by reference.

### STATEMENT REGARDING FEDERALLY SPONSORED RESEARCH OR DEVELOPMENT

Not applicable.

### BACKGROUND OF THE INVENTION

1. FIELD OF THE INVENTION

The present invention relates generally to novel protein modification reagents for fractionation and quantitative (differential) profiling of proteins in a complex mixture. More particularly, the present invention relates to methods of making the protein modification reagents and methods of using the protein modification reagents for quantitative analysis of proteins.

2. DESCRIPTION OF RELATED ART

Proteomics is the large-scale study of proteins, usually by biochemical methods. Traditionally, proteome analysis is accomplished by a combination of two dimensional gel electrophoresis to separate and visualize proteins and mass spectrometry (“MS”) for protein identification.

One approach for protein analysis uses an isotope-coded affinity tag (“ICAT”) See WO 00/11208, “Rapid Quantitative Analysis of Proteins or Protein Function in Complex Mixtures,” which is incorporated herein by reference in its entirety. The reagent consists of biotin for affinity selection, a linker that contains eight light (hydrogen) or heavy (deuterium) isotopes of hydrogen for mass tagging, and a cysteine-reactive group (iodoacetamide) to derivatize proteins. Differential labeling involves using two isotopic reagents for two samples in comparative profiling. Samples are mixed following the ICAT derivatization step, proteolyzed together, tagged peptides are affinity purified using streptavidin, and may be fractionated following extraction from streptavidin prior to mass spectral analysis. The ratio of mass peak amplitude of peptides from proteins differentially labeled with heavy and light mass tags gives a measure of the relative amounts of each protein.

The post-translational modification of proteins is known to be an important mechanism for regulating protein level and activity. Several pathologies have been directly linked to post-translational modifications. For example, ortho modifications of tyrosine, including hydroxylation to DOPA, have been associated with many age-dependent pathologies, including cataracts, Parkinson's disease, amyotrophoic lateral sclerosis, Alzheimers, and atherosclerosis. See Fu et al., Reactions of Hypochlorous Acid with Tyrosine and Peptidyltyrosyl Residues Give Dichlorinated and Aldehydic Products in Addition to 3-Chlorotyrosine, J. Biol. Chem. 273:28,603-28609 (1998); Spencer et al., Intense oxidative DNA damage promoted by L-DOPA and its metabolites: implications for neurodegenerative disease, FEBS Lett 353:246-250 (1994). In addition, 3-nitrotyrosine post-translational modifications have been associated with atherosclerosis, colon cancer, pancreatic cancer, and Alzheimer's disease. See Leeuwenburgh et al., Reactive Nitrogen Intermediates Promote Low Density Lipoprotein Oxidation in Human Atherosclerotic Intima, J Biol Chem 272:1433-1436 (1997); Ambs et al., Frequent Nitric Oxide Synthase-2 Expression in Human Colon Adenomas. Implication for Tumor Angiogenesis and Colon Cancer Progression, Cancer Res 58:334-341 (1998); MacMillan-Crow et al., Tyrosine nitration of c-SRC tyrosine kinase in human pancreatic ductal adenocarcinoma, Arch Biochem Biophys 377:350-356 (2000) Davidsson et al., Proteome analysis of cerebrospinal fluid proteins in Alzheimer patients, Neuroreport 13:611-615 (2002). Unfortunately, ICAT and other general tagging methods are not specific to particular post-translational modifications. In particular, to date, there have been very few reports of a chemically selective methods for the identification of DOPA-proteins or 3-nitrotyrosine proteins in proteomic investigations. Zhang et al., Enrichment and Analysis of Nonenzymatically Glycated Peptides. Boronate Affinity Chromatography Coupled with Electron-Transfer Dissociation Mass Spectrometry, J. Proteome Res., 6, 2257-2268 (2007), reports 3-aminotyrosine was tagged with N-succinimidyl S-acetylthioacetate (“SATA”) at pH 5. This modification allowed for further reaction with hydroxylamine, to yield a free thiol moiety, and subsequent enrichment of the newly formed thiol-containing peptides. However, the published reaction sequence required acetylation of all free amino groups of the peptides prior to reduction of 3-nitrotyrosine to 3-amino tyrosine, and also does not yield a fluorescent entity.

Thus, there remains a need for methods for protein analysis capable of detecting and quantitating levels of post-translational modification, and distinguishing such modified proteins from unmodified proteins. Further, there is a need for analytic methods and reagents that can target native and post-translational modified proteins. Furthermore, there is a need for such reagents that can be synthesized quickly and inexpensively from commercially available materials.

The present invention is directed to methods and reagents to overcome current limitations in traditional analyses performed in proteomics. The reagents can be used as mass labels to provide improved mass spectra of associated analyates.

### BRIEF SUMMARY OF THE INVENTION

The present invention is directed to the identification of a benzylamine derivatization reagents that are specific for 4-substituted catechols and/or 4-substituted 2-aminophenols (reduced nitro-group), which results in the formation of a efficiently fluorescent product, and possesses a molecular structure allowing for preparation of “light” and “heavy” isotopic versions and as a result serve as an enabling basis for relative quantitation by mass spectrometry.

In one aspect, the present invention demonstrated the successful tagging of chemical models for 3-nitrotyrosine (using 2-aminocresol) and hydroxytryptophan (using 5-hydroxyindole). It will be appreciated that the chemical tagging of 3-nitrotyrosine first requires reduction to 3-aminotyrosine, which is accomplished with either dithionite or with suitable metals.

Further, in another aspect, the products resulting from the fluorogenic derivatization of 4-alkyl-catechols and 4-alkyl-2-amino-phenols (reduction product of 2-nitro-phenols) with benzylamine have been conclusively identified. Benzylamine reacts with these substances to form fluorescent benzoxazole derivatives where one benzylamine molecule is involved with oxazole-ring formation and ultimately becomes the 2-substituent, while a second benzylamine molecule becomes a position 6-substituent. These 6-amino-substituted 2-phenylbenzoxazole products provide an explanation for the long-standing observation that many catecholamines (intramolecular 6-amino-substituent) and catechols undergo reaction with benzylamine to form of virtually identical fluorescence characteristics.

Further, in the present invention, it was shown that the fluorogenic benzylamine reaction is applicable in the selective derivatization of DOPA peptides. The 2-phenyl-6- benzylamino-benzoxazole (“BABO”)-modified DOPA residues are amenable to MS analysis and through the use of “light” and “heavy” isotopic forms of BA, relative quantitation appears to be feasible.

Thus, with regard to future application of this chemistry in proteomic investigations of DOPA-proteins and/or 3NY-proteins (reduction to 3-amino-tyrosine required), the incorporation of two benzylamine molecules into the product is potentially a highly favorable result due to the +10 amu shift achieved by use of ²H₅—BA in the derivatization reaction.

It will be appreciated that in certain embodiments of the present invention, the proteins may be first isolated from the sample before they are then labeled with the benzylamine tagging reagent and analyzed by mass spectrometry.

Further, in certain embodiments of the present invention, it is advantageous to separate the proteins in a sample into fractions before tagging and detection. This can be accomplished by a wide variety of methods familiar to those skilled in the art. The separation or fractionation of proteins or peptides may be accomplished by a variety of techniques, including 2-DE, capillary electrophoresis, micro-channel electrophoresis, HPLC, size exclusion chromatography, filtration, polyacrylamide gel electrophoresis, liquid chromatography, reverse size exclusion chromatography, ion-exchange chromatography, reverse phase liquid chromatography, pulsed-field electrophoresis, field-inversion electrophoresis, dialysis, and fluorescence-activated liquid droplet sorting. Alternatively, the proteins or peptides may be bound to a solid support (e.g., hollow fibers (Amicon Corporation, Danvers, Mass.), beads (Polysciences, Warrington, Pa.), magnetic beads (Robbin Scientific, Mountain View, Calif.), plates, dishes and flasks (Corning Glass Works, Corning, N.Y.), meshes (Becton Dickinson, Mountain View, Calif.), screens and solid fibers (see Edelman et al., U.S. Pat. No. 3,843,324; see also Kuroda et al., U.S. Pat. No. 4,416,777), membranes (Millipore Corp., Bedford, Mass.), and dipsticks. If the proteins or peptides are bound to a solid support, within certain embodiments of the invention the methods disclosed herein may further comprise the step of washing the solid support.

In some embodiments it may be desirable to cleave or digest the proteins in a sample, either before or after tagging. This can be accomplished by a wide variety of methods familiar to those skilled in the art. For example, the proteins in the sample may be digested with cyanogen bromide (CNBr) or enzymatically digested (e.g., with trypsin) either before or after being labeled.

A wide range of mass spectrometric techniques also may be useful in the present invention. Representative examples of suitable spectrometric techniques include time-of-flight (TOF) mass spectrometry, quadrupole mass spectrometry, magnetic sector mass spectrometry and electric sector mass spectrometry. Specific embodiments of such techniques include ion-trap mass spectrometry, electrospray ionization (“ESI”) mass spectrometry, ion-spray mass spectrometry, liquid ionization mass spectrometry, atmospheric pressure ionization mass spectrometry, electron ionization mass spectrometry, fast atom bombard ionization mass spectrometry, MALDI mass spectrometry, photo-ionization time-of-flight mass spectrometry, laser droplet mass spectrometry, MALDI-TOF mass spectrometry, APCI mass spectrometry, nano-spray mass spectrometry, nebulised spray ionization mass spectrometry, chemical ionization mass spectrometry, resonance ionization mass spectrometry, secondary ionization mass spectrometry and thermospray mass spectrometry.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 shows the ¹H NMR spectra (aromatic region) for the purified products isolated from the reaction of 4-methylcatechol (a) and 2-aminocresol (b) with benzylamine. These spectra appear to be identical in all respects.

FIG. 2 shows the single crystal x-ray structure determination result for the 4-methylcatechol and benylamine reaction product

FIG. 3 shows the determination of the benzylamine molar excess yield relationship for 4-methylcatechol. Analyte reaction concentration 100 nM with the reaction conducted at 40° C. for a minimum of about 2 hours.

FIG. 4 shows the chromatogram of the 4-methylcatechol or 2-aminocresol product resulting from derivatization with benzylamine. Each analyte was derivatized at the 100 Mn level with 100 mM benzylamine at 40° C. for greater than 10 minutes and subsequently determined by HPLC (details provided herein).

FIG. 5 shows an example chromatogram of equivalent concentration (100 nM analyte in the reaction) 4-methylcatechol solutions derivatized with ¹H₅—BA and ²H₅—BA, then mixed and subjected to HPLC-FI-MS analysis. Mobile phase composition: 9:1 (v/v) solvent B:solvent A (details are provided in the experimental section). The dashed line represents the spectrometer total ion current, and the solid line represents the fluorescence response with excitation/emission settings of 360 nm/475 nm, respectively. The reaction product has an MH+ with an m/z of 315 with ¹H₅—BA and an MH+ with an m/z of 325 with ²H₅—BA. In the structure L represents either ¹H or ²H.

FIG. 6 shows the ion-trap MS/MS of Angiotensin II₃₋₈ converted to the corresponding DOPA-peptide and derivatized with benzylamine to form the BABO derivative (m/z: BABO=Y+206) of the DOPA residue. The identified sequence demonstrates the presence of an intact BABO product after collision induced fragmentation to yield primary sequence information.

FIG. 7 shows the relative quantitation of DOPA-Gly-Gly after derivatization with “light” and “heavy” benzylamine. The chromatograms show the results of mixing the different reaction solutions in the ratios of 1:3, 2:2, 3:1 (L:H; ¹H₅—BA:²H₅—BA), with the panel A chromatograms being filtered for an m/z of 502 and the panel B chromatograms filtered for an m/z of 512. The right portion of each panel illustrates the spectrum for the derivative. LC-MS was conducted with a mobile phase of 8:2 (v/v) solvent B/solvent A.

FIG. 8 shows the mass spectrum of a representative peptide of previously nitrated rabbit phosphorylase b, tagged with benzylamine.

FIG. 9 shows the tagging of nitrated rabbit phosphorylase b with Compound A (BAMS). The various data sets represent tagging efficiencies achieved under variation of the concentration of Fe³⁺. Different isoforms of phosphorylase b were prepared, which contain different amounts of 3-nitrotyrosine, indicated on the x-axis. Tagging efficiency was determined by fluorescence between 460 and 470 nm.

### DETAILED DESCRIPTION OF PREFERRED EMBODIMENT

The present invention is directed to novel protein modification reagents, and the preparation and use of such reagents. These reagents are useful for fractionation and quantitative (differential) profiling of proteins in a complex mixture. The reagents of the present invention are referred to herein as protein mass tag (“PMT”) reagents.

A number of different technologies have been deployed to separate, analyze and identify proteins. Typically, identification by MS involves analysis of isolated proteins or peptide fragments, followed by mapping or tandem MS to obtain sequence information. One strategy that has been used to differentiate the resulting spectra involves tagging the proteins with reagents having different masses (“mass tags”). The use of such mass tags allows a number of different samples to be analyzed at the same time and be directly compared.

The PMT reagents of the present invention comprise a benzylamine moiety that is capable of reacting with a post-translational protein modification. In a preferred embodiment, the post-translational protein modification is selected from the group consisting of 3,4-dihyroxylphenylalanine (“DOPA”), 3-nitrotyrosine (“3-NY”), and hydroxytryptophan (“HO-Trp”). The post-translational modification can occur on an amino acid, a digested peptide or protein fragment or any other protein structure. The adduct of the PMT reagents of the present invention and the protein can be analyzed by mass spectrometry, for example electrospray ionization (“ESI”) MS/MS or matrix assisted laser desorption/ionization (“MALDI”). Proteins originating from different sources can be distinguished based on the mass difference of the PMT reagents. The sequence of the subject proteins can be determined by protein mapping or by tandem mass spectrometry (MS″).

The tagging reagents of the present invention contain a benzylamine functionality, which converts each of the mentioned post-translational modifications into a highly fluorescent benzoxazole entity. Excitation wavelengths are preferably greater than 350 nm (such that they do not interfere with unmodified amino acids in proteins) and emission wavelengths are preferably greater than 450 nm (such that they do not interfere with the known emission wavelengths from unmodified amino acids). More particularly, derivization with benzylamine generates a fluorescent benzoxazole derivatives, which has a fluorescent emission maximum at 460 nm, while derivatization with Compound A and Compound B (see below for structures) generates benzoxazole derivatives with an emission maximum at 480 nm and 510-515 nm, respectively.

The benzylamine tagging agents of the present invention may be modified with one or more accessory moieties can be used to adjust the mass, size, or other physical property of the tagging reagent. The accessory moieties are preferably a functional group which can be utilized for affinity enrichment or improvement of separation of the tagged protein without modifying the tagging procedure itself. Preferred functional groups include —SO₃H and the moiety:

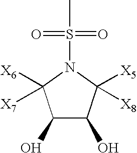

wherein X₅, X₆, X₇, and X₈ are independently H and D.

In some preferred embodiments, the PMT reagent comprises a “recognition group” to aid in the isolation of the labeled protein, such as the cis diol moiety shown in Compound B below.

Suitable recognition groups comprise a detectable moiety, such as, for example, a moiety capable of being isotopically labeled. The detectable moiety can be, for example, a label for detection by an enzyme, a radioactive isotope, a heavier isotope, or a fluorophore, preferably a heavier or lighter isotope. In one aspect, at least 1, 2, 3, 4, 5, 6, 7, 8, 9, or 10 atoms of benzylamine moiety is isotopically labeled. The benzylamine moiety is isotopically labeled such that the mass difference between the labeled and unlabeled regent is sufficiently high to allow for identification of the two forms of a doubly or triple charged peptide with a typical ion-trap resolution. However, the mass difference preferably is not so high that it causes differential retention effects for the peptides. Thus, the reagent preferably incorporates at least about 8 amu mass difference between the heavy and the light forms, preferably about 8 amu to about 100 amu mass difference, more preferably about 8 amu to about 50 amu mass difference, or most preferably about 8 amu to about 20 amu mass difference, or any integer between the stated ranges.

By labeling the proteins with a PMT reagent that comprises a recognition moiety (e.g., biotin), the PMT reagents also serve as a means to obtain selective enrichment of proteins. Use of a recognition moiety is particularly useful when the methods of the invention are applied to proteins that are present in small amounts or when the proteins exist in a complex mixture. In these situations, the recognition moiety can function as a “handle” to allow isolation and concentration of the labeled protein.

The recognition moiety can be any moiety that has an affinity for another species. The list of possible recognition moieties could be expanded to hundreds or thousands of different chemistries, encompassing specific capture agents such as oligonucelotides and/or antibodies as well as ligands for particular receptors, cofactors for proteins, and so forth. It will be appreciated by those skilled in the art that pairs of interacting molecules can be exploited in two ways: (1) with a stationary phase to capture a “ligand” and (2) with a stationary phase to capture a counterligand “receptor.” A list of some but not all types of such pairs in biological systems is listed in Singh et al., U.S. Published Patent No. 2006/0008856 titled “Mass Tags for Quantitative Analysis,” which is incorporated by reference.

It should be understood that countless other examples of specific interactions are known and can be exploited. In this way, for example, the PMT-labeled proteins may be isolated by a streptavidin affinity chromatography and then analyzed by LC/MS. In a simple example, the recognition moiety could be biotin, and the affinity column counterligand could be streptavidin. In another embodiment, the recognition moiety could be a nucleic acid, which could be isolated by hybridization with its complementary sequence.

In a preferred aspect, the benzylamine tagging reagents of the present invention have the structure shown below:

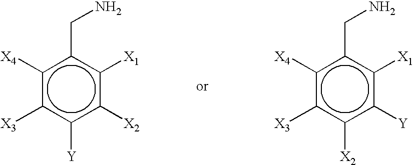

wherein X₁, X₂, X₃, and X₄ are independently H and D; and wherein Y is H or D, or an accessory moiety. While Y may be in the para or meta position, it is anticipated that the ortho position may cause steric hinderance.

In another aspect, the present invention is directed to the benzylamine tagging reagent having the following structure (“Compound A”):

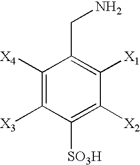

wherein X₁, X₂, X₃, and X₄ are independently H and D.

In one aspect, Compound A allows for the relative quantitation of post-translationally modified peptides/proteins through stable isotope labeling, where one sample is reacted with Compound A wherein X₁, X₂, X₃, and X₄ is H and the other sample is reacted with Compound A wherein X₁, X₂, X₃, and X₄ is D. Both samples can then be analyzed simultaneously by HPLC-mass spectrometry, where the respective quantities are calculated based on the ratio of H to D in a given peptide/protein present in a chromatographic peak.

The —SO₃H group of Compound A usually deprotonates to —SO₃ at pH greater than about 1 so that the tagged protein contains additional negative charges under conditions where peptide enrichment and chromatography are carried out. This renders the tagged proteins more hydrophilic, and shortens their retention time on reverse-phase columns.

In a further aspect, the present invention is directed to a benzylamine tagging reagent having the following structure (“Compound B”):

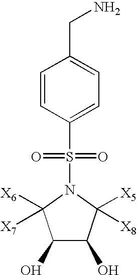

wherein X₅, X₆, X₇, and X₈ are independently H and D.

In one aspect, Compound B represents an affinity tag for selective enrichment by boronate affinity chromatography, permitting sample size reduction and relative quantification of modified peptides/proteins as described for Compound A. Stable isotope labeling for relative quantitation through Compound B is achieved through incorporation of H or D into one of its building blocks, Compound C, where Compound C also represents a novel molecule. Thus, in still another aspect, the present invention is directed to an intermediate compound according to the following structure (“Compound C”):

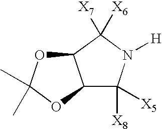

wherein X₅, X₆, X₇, and X₈ are independently H and D.

Pathway for Formation of Benzoxazoles From Reaction of Catechols and o-Aminophenols With Benzylamine Tagging Agents

As discussed more fully below, the reaction product of benzylamine and 4-methylcatechol and 2-aminocresol was investigated. The identification of the reaction product as benzoxazole 9 allows one to propose a rational reaction pathway for the derivatization of 4-substituted catechols and 4-substituted 2-amino-phenols with benzylamine. The derivatization reaction is typically conducted at basic pH in the presence of an oxidant such as potassium hexacyanoferrate K₃Fe(CN)₆ and the primary amine nucleophile (benzylamine). A detailed pathway for reaction of these analytes with benzylamine is illustrated in the scheme below. Initially the analytes are oxidized to the corresponding o-quinoid intermediate 5, followed by a 1,4-reductive Michael addition to form the benzylamino-substituted intermediate 10, which is further oxidized to the corresponding o-quinoid intermediate 11. Intermediate 11 undergoes a condensation reaction with a second benzylamine molecule at the most electrophilic carbonyl and a subsequent imine isomerization leads to the Schiff base 13. Schiff base 13 is likely in equilibrium with the dihydrobenzoxazole 14, which leads to the formation of product 9 by an irreversible oxidative step. The in situ generation of quinones that undergo reaction with nitrogen nucleophiles, ultimately leading to the formation of an amino-substituted quinone such as structure 11 is well established and is responsible for the formation of adrenochrome (autooxidation of epinephrine). Once formed, quinones similar to 11 have been established to undergo reaction with benzylamine under mild oxidative conditions to ultimately yield benzoxazoles similar to 9 via the sequence shown.

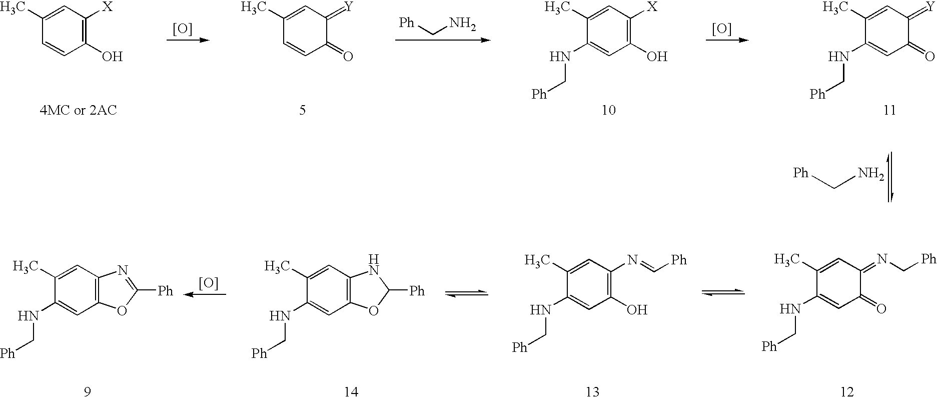

wherein X is —OH and Y is O for 4-methylcatechol; and

wherein X is —NH₂ and Y is NH for 2-aminocresol.

### Definitions

In describing the present invention, the following terms will be employed, and are to be defined as indicated below.

A “protein” or a “polypeptide” is used in it broadest sense to refer to a compound of two or more subunit amino acids (e.g. 2, 3, 4, 5, 6, 7, 8, 9, 10, 11, 12, 13, 14, 15, 16, 17, 18, 19, 20 residues) or amino acid analogs. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids (usually up to about 40 amino acids) is commonly called an oligopeptide if the peptide chain is short. If the peptide chain is long, the peptide is typically called a polypeptide or a protein. Full-length proteins, analogs, and fragments thereof are encompassed by the definition. The terms also include postexpression modifications of the polypeptide, for example, glycosylation, acetylation, phosphorylation, ubiquitination, and the like. Furthermore, as ionizable amino and carboxyl groups are present in the molecule, a particular polypeptide may be obtained as an acidic or basic salt, or in neutral form. A polypeptide may be obtained directly from the source organism, or may be recombinantly or synthetically produced.

As used herein, a “biological sample” refers to a sample of tissue or fluid isolated from a subject. Typical samples include but not limited to, blood, plasma, serum, fecal matter, urine, bone marrow, bile, spinal fluid, lymph fluid, samples of the skin, secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells and cell components. Thus, biological samples include not only samples obtained from living organisms (e.g., mammals, fish, bacteria, parasites, viruses, fungi, and the like) or from the environment (e.g., air, water, or solid samples), but biological materials which may be artificially or synthetically produced (e.g., phage libraries, organic molecule libraries, pools of genomic clones, and the like). Representative examples of biological samples include biological fluids (e.g., blood, semen, cerebral spinal fluid, urine), biological cells (e.g., stem cells, B or T cells, liver cells, fibroblasts, and the like), and biological tissues.

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, strepavidin or haptens), and the like.

As used herein, a “solid support” refers to a solid surface such as a magnetic bead, latex bead, microtiter plate well, glass plate, nylon, agarose, acrylamide, and the like.

Sample Preparation

In this aspect of the invention, the protein can be obtained from essentially any source. The protein can be isolated and purified to be free of interfering components. In one aspect of the invention, the protein is “substantially pure,” which means that the protein is about 80% homogeneous, and preferably about 99% or greater homogeneous. Many methods well known to those of ordinary skill in the art may be utilized to purify the protein prior to its digestion or prior to determining its amino acid sequence. Representative examples include HPLC, Reverse Phase-High Pressure Liquid Chromatography (RP-HPLC), gel electrophoresis, chromatography, capillary electrophoresis, immobilized metal affinity chromatography (IMAC), or any of a number of peptide purification methods.

Protein Digestion

In one aspect of the invention, the proteins are contained in a biological samples or may be recombinantly or synthetically produced. The proteins may be digested with any of the well-known protein digestion reagents. Such reagents may be chemical or enzymatic.

The range of protein cleavage techniques include digestion by proteases including papain, clostropain, trypsin, LysC, GluC, and by chemical digestion including limited acid digestion, and cyanogen bromide.

Proteases useful in practicing the present invention include without limitation trypsin, chymotrypsin, V8 protease, elastase, carboxypeptidase, papain, pepsin, proteinase K, thermolysin, and subtilisin (all of which can be obtained from Sigma Chemical Co., St. Louis, Mo.). The protease for use in practicing the present invention is selected such that the protease is capable of digesting the particular target protein under the chosen incubation conditions. Papain cleaves on the carboxy-terminal side of Arg-X, Lys-X, His-X, and Phe-X, and is a relatively mild protease, is commercially available in a highly purified form, and is available attached to solid supports (Sigma). The advantage of using a protease attached to a solid support is that this allows the complete and easy removal of the protease following digestion. Clostropain cleaves on the carboxy-terminal side of arginine residues, and is preferably used if the preferred cleavage site is Arg-Tyr. Trypsin is most commonly used for protein digestion, and cleaves on the carboxy-terminal side of arginine and lysine residues. However, if larger fragments are preferred, LysC can be used to digest the protein. LysC only cleaves at lysine residues, therefore, on average produces larger fragments than trypsin.

In one aspect, the peptides may range in size from one amino acid to 50 or more, preferably about 5 amino acids to about 20 amino acids, depending on the protein sequence and the type of mass spectrometer to be used for analysis. Thus, the molecular weight for such peptides is from about 50 to 20,000 daltons. The molecular weight of the peptides for use in the invention is preferably about 200 amu to about 3000 amu, more preferably about 300 amu to about 1500 amu.

Tagging the Proteins and Peptides

Typically, a target peptide or protein is first taken up to a final concentration of 1-100 μg/ml in an aqueous solution, preferably containing some methanol (up to 90% v/v, if solubility allows).

The tagging of proteins with various benzylamine reagents described and disclosed above in an aqueous or mixed aqueous/organic solvent may be employed.

In general, the peptide or the mixture of peptides is contacted with benzylamine tagging reagent in a suitable solvent, preferably a water/organic solvent system comprising water and methanol, and most preferably 90:1 methanol water. Other suitable organic solvent, such as, for example, DMSO, DMF, xylenes, acetone, and the like, may be employed.

In the case of 3-NT proteins, the peptides are first reduced to 3-aminotyrosine peptides. This may be performed using dithionite according to Viner et al., Protein modification during biological aging: selective tyrosine nitration of the SERCA2a isoform of the sarcoplasmic reticulum Ca2+-ATPase in skeletal muscle, Biochem. J., 340, 657-669 (1999).

Next, the proteins are oxidized using a suitable oxidizing agent. The preferred oxidizing agent is an iron containing compound, such as K₃CN₆Fe.

The concentration of protein is preferably between about 1 and 10 μmol/L, the concentration of benzylamine is preferably between about 1 mM and 1.0 M, and the concentration of the oxidizing agent is between about 10 μM and 5 mM.

Sequence and Identity Determination

The methods of the present invention are utilized in order to determine the sequence and/or identity of a protein. In one aspect of the invention, the tagging procedure is performed on a mixture of peptides. Following the tagging procedure the mixture of peptides is submitted to a separation process, which preferably, allows the separation of the protein mixture into discrete fractions.

In the methods of the present invention, the peptides tagged with the benzylamine tagging reagents are sequenced by a mass spectrometer. Various mass spectrometers may be used within the present invention. Representative examples include, triple quadrupole mass spectrometers, magnetic sector instruments (magnetic tandem mass spectrometer, JEOL, Peabody, Mass.); ion-spray mass spectrometers; electrospray mass spectrometers; laser desorption time-of-flight mass spectrometers; quadrupole ion-trap spectrometers; and a Fourier Transform Ion Cyclotron Resonance Mass Spectrometer (Extrel Corp., Pittsburgh, Pa.). In one aspect of the invention, an electrospray mass spectrometer (Agilent Technologies, Palo Alto, Calif.) is utilized to fragment the tagged peptides, and a time-of-flight detector with better than 50 ppm mass accuracy is used to determine the sequence from the masses of the labeled fragments.

Although substantially pure peptides are preferably utilized within the methods described herein, it is also possible to determine the sequence of peptide mixtures. Briefly, in one aspect, an algorithm is utilized in order to determine all of the hypothetical sequences with a calculated mass equal to the observed mass of one of the peptides in the mixture (see Johnson et al., Sequence analysis of peptide mixtures by automated integration of Edman and mass spectrometric data, Protein Science 1:1083-1091 (1992)). These sequences are then assigned figures of merit according to how well each of them accounts for the fragment ions in the tandem mass spectrum of the peptide. Utilizing such algorithms, the sequence of polypeptides within the mixture may be readily determined.

One of skill in the art will appreciate that the sequence information obtained using the methods of the invention can be combined with other characteristics of the protein under analysis to even further reduce the number of possible identities of the protein. Thus, in a preferred embodiment, the method of the invention combines information from a protein sequence tag with one or more other protein characteristics to identify the protein. Data that are useful to supplement the sequence data include, but are not limited to, amino acid composition, the number and identity of specific residues (e.g., cysteine), cleavage information, proteolytic (e.g., tryptic) and or chemolytic peptide mass, subcellular location, and separation coordinates (e.g., retention time, pI, 2-D electrophoresis coordinates, etc.). Other forms of data characteristic of a particular protein or class of proteins that can be combined with information from use of the compositions and methods of the invention to identify a protein will be apparent to those of skill in the art. As the body of data characteristic of a particular protein becomes more comprehensive, proteins under analysis can be identified using shorter sequences.

Differential Expression

In one aspect of the invention, the benzylamine tagging compounds are used in methods for detecting the differential expression of the same protein in two samples, or the presence of protein(s) in some, but not all, samples. The present invention thus provides methods of identifying one protein or more than one protein, that are differentially present in samples. The samples, as defined above, include, for example, sample from a healthy patient and a samples from a diseased patient. When the samples are biological specimens, the information provided by the inventive methods may be used to determine which protein(s) are differentially expressed in the samples. As used herein, the term “differentially present” means that one or more proteins is present at a higher relative amount in one of the samples as compared to the rest of the samples. The term also means that protein(s) are present in one of the samples that are not present in the rest of the samples.

In one aspect of the present invention, peptides from two or more samples are analyzed separately, or the peptide mixtures can be combined for analyses in a single analytical run. The peptide mixtures can be obtained from different sources and the peptides are then differentially labeled. The use of differential labeling for the two samples yields one sample peptide mixture with a characteristic label, such as, for example, a benzylamine tag of the invention, whereas the peptides in the other sample mixture bear a different characteristic label, such as, for example isotopically labeled U-PIT tag. Once the mixtures are combined and then subjected to analysis by mass spectrometric means described above, variations in the ratio of signals from the two labels indicates different amounts of that particular peptide, and, thus, differential expression of the precursor protein.

The compositions and methods of the invention are thus useful for identifying proteins from a healthy or a diseased tissue sample. In one aspect, the compositions and methods are applied to both a mixture of proteins from a healthy tissue sample and a mixture of proteins from a diseased tissue sample. The samples can then be analyzed individually or as a mixture, as described above.

In another aspect of the invention, the compositions and methods of the invention are applied to a plurality of samples where each sample can contain a single protein or a mixture of proteins. In this aspect of the invention, each sample is labeled with a selected benzylamine tagging reagent of the invention which differs from the benzylamine tagging reagent used for all other samples by at least 1 mass units. The difference in mass is preferably achieved by differentially isotopically labeling the benzylamine tagging reagent. The compositions and methods of the invention thus find use in proteomics.

Kits

The subject sets of benzylamine tagging reagents may be sold in kits, where the kits may or may not comprise instructions for use, and additional reagents or components necessary for the particular application in which the benzylamine tagging reagent is to be employed. Thus, for sequencing applications, the sets may be sold in a kit which further comprises one or more of the additional requisite sequencing reagents, such as peptide digestion enzymes, polymerases, nucleotides, buffers, separation columns for particular peptides or proteins, such as affinity columns or IMAC columns, software, and the like.

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Overview of Synthesis of Exemplary Benzylamine Tagging Reagents In Examples 1-7

As discussed in Examples 1 to 7 below, benzylamine tagging reagents of the present invention were prepared according to the following scheme:

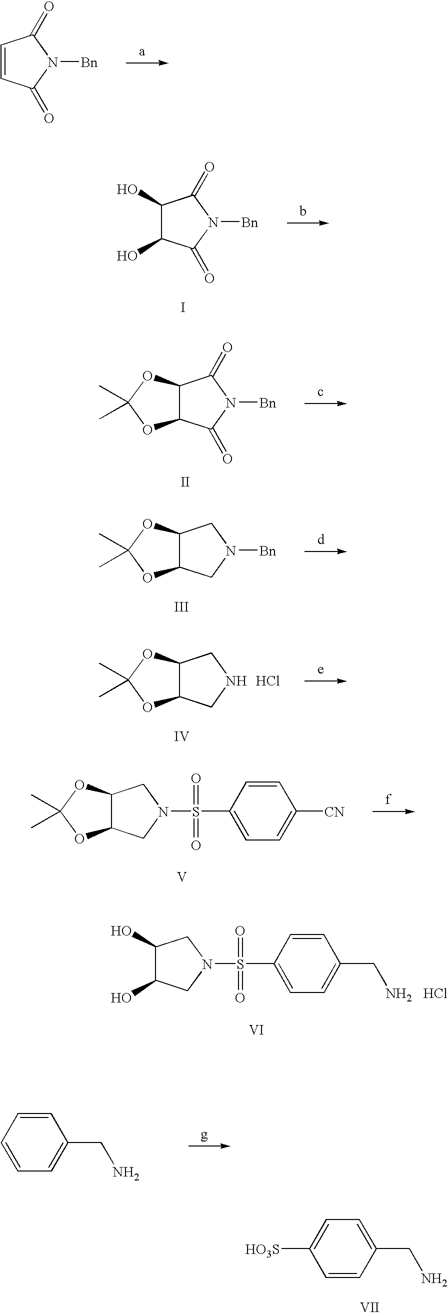

### EXAMPLE 1

### Synthesis of N-benzyl-(3R, 4S)-3,4-dihydoxypyrrolidine-2,5-dione (Compound I)

N-benzylmaleimide was prepared by reaction of benzylamine with maleic anhydride followed by sodium acetate in acetic anhydride (45%). To a solution of 4.5 g of N-methylmorpholine N-oxide and 6 ml of osmium tertraoxide solution (2 wt % in tert-butyl alcohol) in 50 ml of acetone and 10 ml of distilled water was added 6.0 g N-benzylmaleimide. The mixture was stirred at 45° C. for 24 hours. After the reaction mixture was cooled to room temperature, 2.0 g NaHSO₃ was added and stirred for 30 minutes, and filtered through a sintered glass funnel. The filtrate was concentrated by evaporation under reduced pressure and extracted twice with 50 ml of ethyl acetate. The combined organic extract was dried over Na₂SO₄ and evaporated under reduced pressure, affording deep colored gum which was allowed to granulate for 4 hours at room temperature. The resulting solid that formed was washed with minimum volume of ethyl acetate and dried in vacuum to give compound I (5.2 g, 73% yield) as white powder. Mp 125-127° C.; NMR (DMSO-d₆): δ 6 7.3 (m, 5 H), 6.02 (d, 2 H), 4.55 (s, 2 H), 4.42 (d, 2 H).

### EXAMPLE 2

### Synthesis of N-benzyl-(3aR, 6aS)-dihydro-2-dimethyl-4H-1,3-dioxolo[4,5-c]pyrrole-4,6(5H)-dione

### (Compound II)

To a slurry of 8.5 g of I in 40 ml of 2,2-dimethyloxyprpane was added 0.02 g of p-toluenesulfonic acid and the mixture was stirred at room temperature for 10 hours. The reaction mixture was then stirred with 0.5 g of sodium bicarbonate for 10 minutes, filtered, and concentrated by evaporation under reduced pressure and the residue was granulated at room temperature. The solid that formed was washed with petroleum ether and dried under vacuum overnight to provide the product 11 (9.6 g, 95% yield) as white powder. NMR (CDCl₃): δ 7.32 (m, 5 H), 4.86 (s, 2 H), 4.69 (s, 2 H), 1.43 (s, 3 H), 1.30 (s, 3 H).

### EXAMPLE 3

### Synthesis of N-benzyl-(3aR, 6aS)-tetrahydro-2,2-dimethyl-4H-1,3-dioxolo[4,5-c]pyrrole (Compound III)

To a solution of 9.5 g of compound II in 20 ml of diethyl ether was added in portion 5.0 g of LiAlH4. The slurry was heated to reflux at 80 ° C. for 24 hours and cooled in ice-bath. To the reaction mixture were slowly added 30 ml of ethyl acetate and then 20 ml of 30% NaOH solution with stirring. After 30 minutes, the mixture was filtrated, the filter cake was washed twice with 30 ml of ethyl acetate. The combined organic layer was dried over Na2SO4 and evaporated under reduced pressure to give product as oil. Yield: 8.2 g, 92%. NMR (CDCl₃): ε 7.32 (m, 5 H), 4.66 (s, 2 H), 3.63 (s, 2 H), 3.0 (d, 2 H), 2.1 (d, 2 H), 1.59 (s, 3 H), 1.34 (s, 3 H).

### EXAMPLE 4

### Synthesis of (3aR,6aS)-tetrahydro-2,2-dimethyl-4H-1,3-dioxolo[4,5-c]pyrrole (Compound IV)

To a solution of 8.0 g of compound III in 50 ml of methanol was carefully added 0.8 g of 10% Pd/C catalyst. The slurry mixture was hydrogenated at 40 psi for 18 hours at room temperature, and filtered to remove catalyst, and the filter cake was washed with 20 ml of methanol. The combined organic solution was evaporated under reduced pressure. The residue was dissolved in 50 ml of ethyl ether, and 10 ml of 1.0 M HCl in ether was added. The precipitate that formed was collected by filtration, washed with ether and dried under vacuum to give compound IV (5.42 g, 89%) as white powder. NMR (CDCl₃): δ 4.98 (s, 2 H), 4.87 (s, 2 H), 3.60 (d, 2 H), 3.30 (d, 2 H), 1.52 (s, 3 H), 1.35 (s, 3 H).

### EXAMPLE 5

### Synthesis of (3aR,6aS)-tetrahydro-2,2-dimethyl-4H-1,3-dioxolo[4,5-c]pyrrole-5-(4H,6H,6aH)ylsulfonylbenonitrile (Compound V)

To a solution of 3.0 g of IV in 50 ml of acetonitrile was added 5.0 ml of triethylamine and 3.4 g of 4-cyanobenzene-1-sulfonyl chloride, and the mixture was stirred for 1 hour and filtered. The filtrate was washed with 10 ml of saturated aqueous solution of NaCl, and evaporated under reduced pressure to give product V (5.6 g, 89% yield) as pale yellow solid. NMR (DMSO-d₆): δ 8.39 (d, 2 H), 8.03 (d, 2 H), 4.69 (s, 2 H), 3.67 (d, 2 H), 3.05 (d, 2 H), 1.32 (s, 3 H), 1.30 (s, 3 H).

### EXAMPLE 6

### Synthesis of (3R,4S)-1-(4-(aminomethyl)phenylsulfonyl)pyrrolidine-3,4-diol (Compound VI)

To a solution of 5.0 g of compound III in 50 ml of methanol was carefully added 0.5 g of 10% Pd/C catalyst. The mixture was hydrogenated at 40 psi for 18 hours at room temperature, and filtered to remove catalyst, and the filter cake was washed with 20 ml of methanol. The combined organic solution was evaporated under reduced pressure. To the residue was added 40 ml of 2 M HCl aqueous solution, and heated at 80° C. and stirred for 2 hours. The solvent was removed by evaporation under reduced pressure to give product VI as white powder. NMR (DMSO-d₆): δ 7.86 (d, 2 H), 7.73 (d, 2 H), 4.14 (d, 2 H), 3.86 (s, 2 H), 3.35 (d, 2 H), 3.00(d, 2 H).

### EXAMPLE 7

### Synthesis of 4-Sulfobenzylamine (Compound VII)

10 ml of benzylamine was added dropwise at 0° C. to 30 ml of 20% fuming H₂SO₄ with stirring. The mixture was heated to room temperature and stirred for 30 minutes and then to 80 ° C. for 1 hour. After cooling to room temperature, the reaction mixture was poured into 400 ml of cool dioxane. The solid that formed was collected by filtration through sintered glass funnel, and washed with 50 ml of cool dioxane. The compound was purified by dissolving in a minimum volume of ammonia solution and then precipitated upon addition of concentrated HCl until ph 1 affording 5.5 g product. NMR (DMSO-d₆): δ 8.11 (br, s, 3 H), 7.63 (d, 2 H), 7.38 (d, 2 H, 4.02 (s, 2 H).

Materials and Methods in Examples 8-16

Reagents and Solvents

In the following examples, benzylamine (BA), epinephrine (E), formic acid (FA), meso-1,2-diphenylethylamine (DPE), 4-methylcatechol (4-methylcatechol), mushroom tyrosinase (1530 U/mg), Tyr-Gly-Gly, angiotensin II₃₋₈ and potassium hexacyanoferrate (III) (K₃Fe(CN)₆) were obtained from Sigma-Aldrich (St. Louis, Mo.) and used as received. The following solvents (all HPLC grade): acetone, acetonitrile (ACN), 95% ethanol (EtOH) ethyl acetate (EtOAc), hexanes (Hx), methanol (MeOH), and methylene chloride (CH₂Cl₂) were obtained from Fisher Scientific (Pittsburg, Pa.). The benzylamine isotopes, ²H₅-benzylamine (²BA, 99% isotopic enrichment) and ²H₇-benzylamine (²H₇—BA, 99% isotopic enrichment) hydrochloride were obtained from CDN isotopes (Quebec, CAN), and ¹⁵N-benzylamine (¹⁵N—BA, 99% isotopic enrichment) was purchased from IsoTech (Miamisburg, Ohio). For NMR experiments, d₆-acetone was used as the solvent (Fisher Scientific). Purified water was obtained using a Labconco Water ProPS Filtration System (Labconco Corporation, Kansas City, Mo.).

HPLC-Fluorescence Detection

All separations were conducted utilizing a GL Science Inertsil ODS-3 (4.6 mm i.d.×150 mm, 5 μm spherical particles, 100 Å pore size) column. For all separations the mobile phase was 0.1% FA in water (solvent A) and 0.1% FA in ACN (solvent B). A modular system was assembled consisting of the following Shimadzu components: SCL-6B system controller, Sil-6B autosampler (equipped with a temperature controlled sample holder), two LC-6A pumps, CTO-6A column heater, SPD-10AV UV detector and RF-535 fluorescence detector set at 360 nm excitation and 475 nm emission. The column temperature was held at 40° C., injection volumes were 50 μL, and a 0.7 mL/min flow rate was utilized for all separations.

HPLC-Fluorescence/MS Detection

The separation column, mobile phase composition and flow rate, and fluorescence detection parameters were as previously noted for HPLC with fluorescence detection. Separation with dual detection (fluorescence/MS) was accomplished with the following system: a Waters Associates Alliance 2690 Chromatographic System, Shimadzu RF-535 fluorescence detector and a MicroMass QuattroMirco™. All separations with this system were conducted at ambient temperature utilizing 25 μL injections unless otherwise noted.

Preparative Chromatography

Low pressure preparative separations were conducted using an apparatus consisting of a Beckman 110B Solvent Delivery Module, and glass columns with screw on end-fittings (25 mm i.d.×450 mm; Ace Glass, Vineland, N.J.). Silica gel (200-400 mesh, 60 Å pore; Sigma-Aldrich) was used for all separations. Injections were accomplished manually by using a syringe with mobile phase flow stopped.

Instrumentation and Apparatus

Fluorescence excitation and emission spectra were acquired using a Jasco (Easton, Md.) FP-6500 fluorescence spectrometer. DSC was conducted with a Model Q100 DSC (TA Instruments-Waters LLC, New Castle, Del.). NMR experiments were conducted on a Bruker 500 MHz instrument equipped with a dual carbon/proton CPDUL cryoprobe. X-ray crystal structure determination was performed using a Bruker AXS SMART APEX CCD diffractometer (Bruker-AXS, Madison, Wis.) by the X-Ray Crystallography Laboratory at the University of Kansas (operational and structure solution details are available upon request). Ion-Trap MS experiments were conducted by direct infusion into a Thermo Finnigan LCQ Ion-trap MS (Thermo Fisher Scientific Inc., Waltham, MA) with the aid of a Picotip™ emitter from New Objective (Woburn, Mass.).

Derivatization Investigations

Reactions were conducted according to a previously described procedure in Nohta et al., Aromaticglycinonitriles and methylamines as pre-column fluorescene derivatization reagents forcatecholamines, Anal. Chim. Acta. 344:233-240 (1997), with slight modifications. In general, to 700 μL of 9:1 (v/v) MeOH:H₂0, was added in order, 100 μL of 1.0 μM analyte (50, 100, 250, 500, and 1000 nM for the fluorescence calibration plot), 100 μL of 50 mM aqueous K₃Fe(CN)₆, and 100 μL of reagent solution (BA, ²H₅—BA, ²H₇—BA, ¹⁵N—BA or DPE). For benzylamine excess-yield experiments, reagent concentrations ranged from 0.001 M to 1.0 M aqueous solutions in one-half decade concentration increments. For analytical derivatizations, all isotopic benzylamine reagents were prepared as 1.0 M aqueous solutions while the DPE reagent was a 0.1 M EtOH solution. In each case, after the various additions were completed, the reaction vial was sealed, quickly shaken and stored at 40° C. The reagent excess-yield experiments were kept for approximately 2 hours then analyzed by HPLC with fluorescence detection. Kinetic-yield experiments with benzylamine (analytical derivatization protocol) were conducted in a similar fashion, with reaction yields being determined at various timed intervals up to 40 minutes.

### EXAMPLE 8

### Tagging 4-Methylcatechol with Benzylamine

In this example, 4-methylcatechol was tagged with benzylamine. More specifically, to 250 mL of an 50% aqueous methanolic solution of 4-methylcatechol (2.0 g, 16.1 mmol) was added K₃Fe(CN)₆ (10.0 g, 30.4 mmol) with stirring until dissolution was complete. Subsequently, benzylamine (25 mL, 229 mmol) was added dropwise with stirring over a 30 minute period. The reaction was stirred at ambient temperature for an additional 45 minutes, then gently heated to evaporate the majority of the methanol. The resulting aqueous solution was extracted with methylene chloride, the organic layer recovered, dried with anhydrous magnesium sulfate, filtered and evaporated under reduced pressure to yield a reddish gummy residue. A portion of this residue (0.65 g) was taken up in ethyl acetate (10 mL) and purified by low-pressure preparative chromatography. Collection of the appropriate fraction followed by solvent evaporation yielded a white solid, which was recrystallized from acetone/water to yield needle shaped crystals, m.p. 143° C. (uncorrected). To obtain crystals suitable for x-ray diffraction studies, the initially obtained crystals were recrystallized by dissolution in acetone with and exposure to hexane vapor (separate container) with each vessel being sealed in a larger container for approximately one week. A slow crystallization process ensued that provided the desired larger crystals. For x-ray diffraction studies, the crystals were left in the mother liquor and passed on to the University of Kansas X-ray crystallography facility. When a portion of these crystals were recovered and dried at 80 ° C. for a short period, DSC analysis provided a m.p. 143° C. This product, N-benzyl-5-methyl-2-phenylbenzo[d]oxazol-6-amine, was utilized for all NMR experiments.

Interestingly, utilizing 4-methylcatechol as the catechol and benzylamine as the derivatization reagent, the results obtained were in conflict with previous reports. MS characterization of the product formed from reaction of benzylamine with 4-methylcatechol revealed a molecular ion of MH+=315 instead of the expected MH+=210. Continuing experimentation using various isotopic forms of benzylamine provided the following results: 2 H₅—BA, observed MH+=325 (expected 215); ¹⁵N—BA, observed MH+=317 (expected 211); 2 H₇—BA, observed MH+=327 (expected 215).

Noting that the molecular weight of benzylamine is 107, examination of these data reveal a consistent pattern. If an additional benzylamine moiety (C₆H₅CH₂NH—) was substituted anywhere on the benzoxazole ring system or a similar isobaric structure, then a net mass increase of +105 Da would result. In other words, two equivalents of benzylamine appear to be involved in the derivatization of 4-methylcatechol (a catechol) as compared to only one benzylamine molecule in the derivatization of epinephrine (a catecholamine). Referring to the □M column with +105 Da as the reference point, the +10 Da and +12 mass shift from use of ²H₅—BA and ²H₇—BA, respectively allow one to speculate that during product formation one of the benzylamine molecules loses both benzylic hydrogens, while the second benzylamine molecule retains these hydrogens. The +2 Da increase from use of ¹⁵N—BA in the derivatization of 4-methylcatechol and 2-aminocresol (see below) further reveals a product is formed containing two nitrogens originating from the benzylamine reagent. Overall, these data are consistent with the formation of a product having an empirical formula of C₂₁H₁₈N₂O (MW=314).

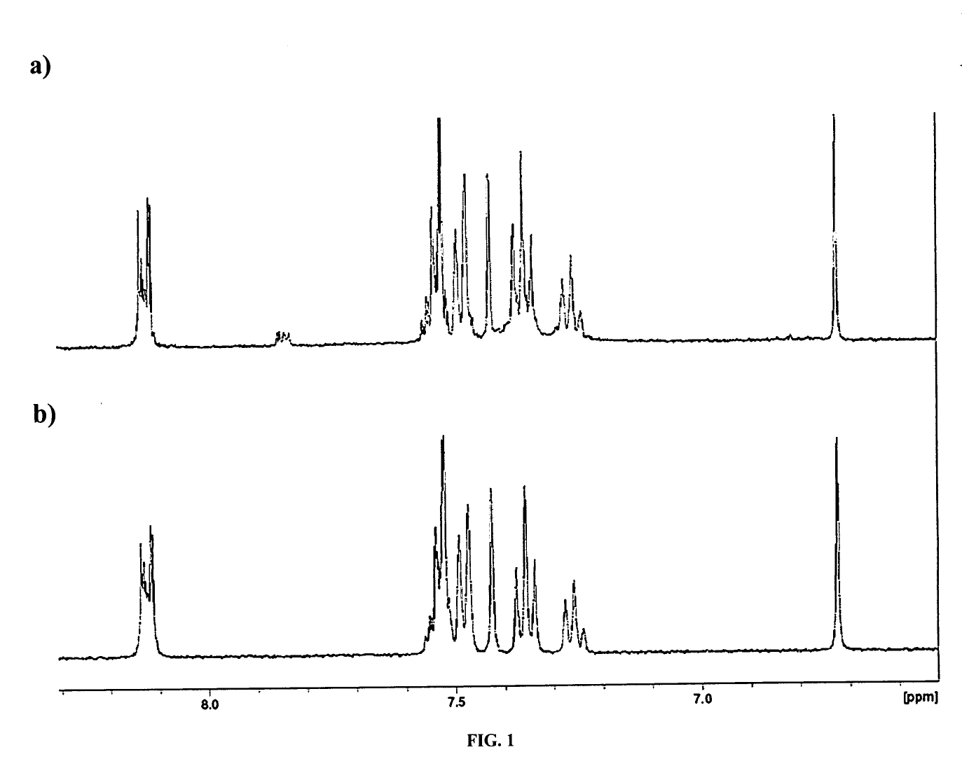

The decision was made to scale-up the derivatization reaction in order to expedite the isolation and purification of the reaction products. Following a procedure similar to that previously described by Nohta et al., Anal Chim Acta 344:233-240 (1997), this was completed for 4-methylcatechol and 2-aminocresol (see below). In each case crystalline products were isolated that exhibited the same HPLC retention time, parent ion MS (MH+=315) and fluorescence characteristics (excitation/emission max, 344 nm/467 nm, respectively) as previously observed from the in situ product generation investigations. Recrystallization provided a high purity standard that was subjected to a series of NMR experiments and single x-ray structural analysis. The experimentally obtained ¹H NMR spectra were identical in all respects for each product and was supportive of a structure possessing 12 aromatic protons, 2 benzyl protons and 3 methyl protons. Such resonances were observed in FIG. 1 as singlets at 6.72 ppm and 7.42 ppm, respectively. Calculated spectra (ACD Labs HNMR/CNMR Predictor®; Advance Chemistry Development, Inc., Toronto, CAN) predicted resonances of 6.65 ppm (position 7) and 7.39 ppm (position 4) for the proposed reaction product according to the above scheme. Additional more sophisticated NMR experiments were subsequently conducted (¹3C NMR and 2 D NMR), whose results were consistent with the product possessing structure set forth in the scheme.

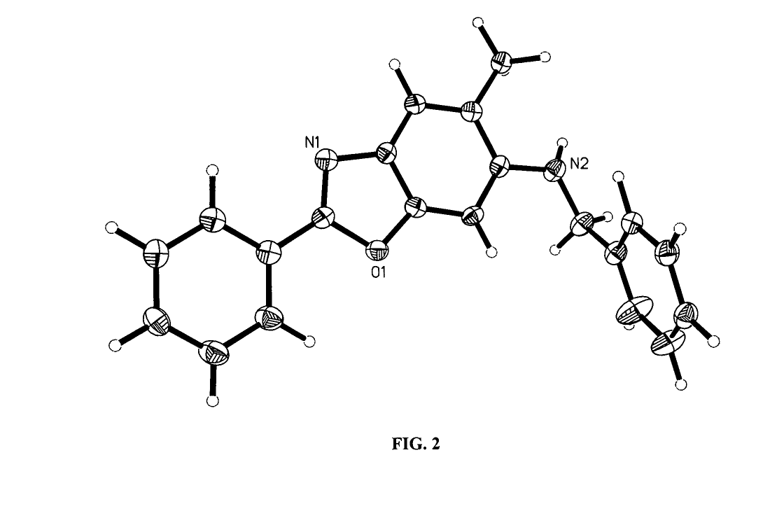

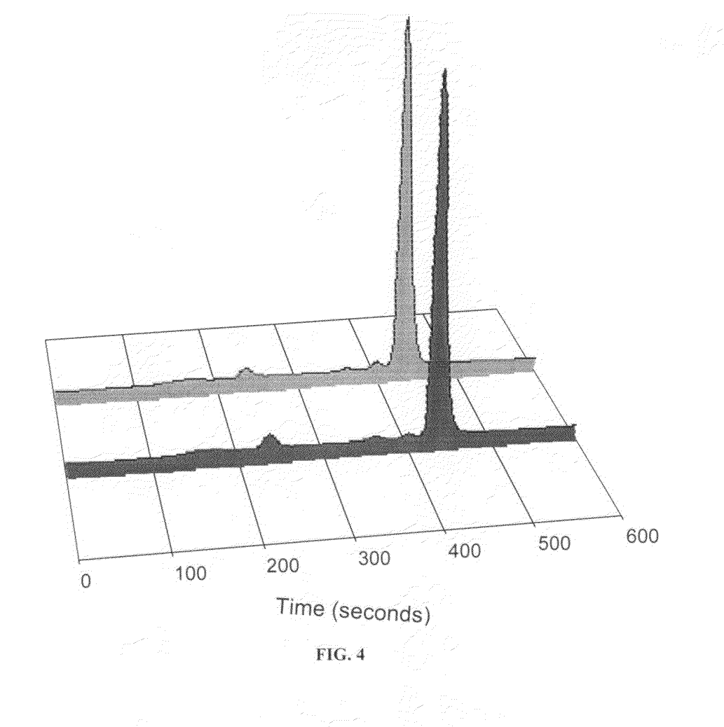

While the NMR experiments were ongoing, an x-ray structural determination was performed. The structure obtained (FIG. 2) was in complete agreement with the various NMR experiments to definitively prove that N-benzyl-5-methyl-2-phenylbenzo[d]oxazol-6-amine, is the fluorescent product obtained from reaction of 4-methylcatechol with benzylamine. Further, the chromatograms shown in FIG. 4 illustrate the identical yields of product obtained from the derivatization of either 4-methylcatechol and 2-aminocresol with benzylamine at the 100 nm level.

Of significance was the establishment by chromatographic retention, fluorescence spectral and NMR experiments that 2-aminocresol undergoes reaction with benzylamine to form that same structure (see Example 9), thus in addition to use as a DOPA-peptide reagent, benzylamine appears to provide a pathway for the selective derivatization of 3NY-peptides if initially reduced to 3AT-peptides.

### EXAMPLE 8A

### Investigation of Benzylamine Concentration in Tagging 4-methylcatechol

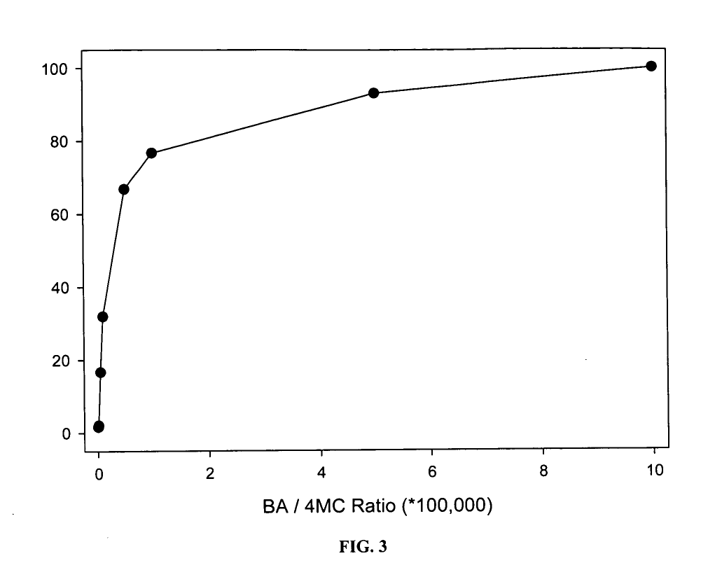

Example 8 illustrates a simple derivatization protocol was adopted that did not require a buffer. A MeOH/H₂0 solution serves as the reaction solvent, with analytes, oxidant and benzylamine being added as aqueous solutions. Using this procedure, experiments were directed towards defining the required excess of benzylamine as regards the reaction yield and rate for 4-methylcatechol at a specified temperature (40° C.). When the benzylamine concentration was varied over a 10³-fold to 10⁶-fold excess, it was found that the yield is essentially invariant once a molar excess of 10⁵ was reached (FIG. 3). Regarding the present investigation, these results were obtained from analysis of the reaction solutions after a 2 hour period. In a separate experiment, the same benzylamine excess concentration effect was again probed, but the reaction was conducted overnight with approximately 18 hours elapsing prior to product determination. Virtually identical results were obtained in this extended experiment, which was not unexpected since earlier investigations had shown the product to be stable for several days in the derivatization solution (data not presented). As a result, an analytical derivatization protocol was adopted utilizing benzylamine at a concentration of 0.1 M (ideally ≧10⁵-fold excess with respect to the analyte). Experiments were then conducted to define the kinetic course of the 4-methylcatechol and 2-aminocresol reactions at 40° C. When these derivatization reactions were performed with the analytes at 150 nM (40° C.) maximal yield was obtained within 10 minutes and continued determinations of these solutions over a 40 minute period indicated normalized relative yields of 100.1±0.42% for 4-methylcatechol and 100.0±1.99% for 2-aminocresol, respectively.

Together these experiments indicate an interesting result. Maximal product yield requires a large excess of BA; however, product formation in high yield occurs in a relatively short time frame. This is suggestive that the in situ generated nascent quinone (or iminoquinone) partitions between productive (see scheme) and non-productive pathways (unknown) as regards the formation of the product. If operative such a situation explains the need for the large excess of benzylamine to achieve maximal yield, i.e., kinetic steering towards the productive reaction pathway.

### EXAMPLE 8B

### Relative Ouantitation of the Benzylamine and 4-methylcatechol Product

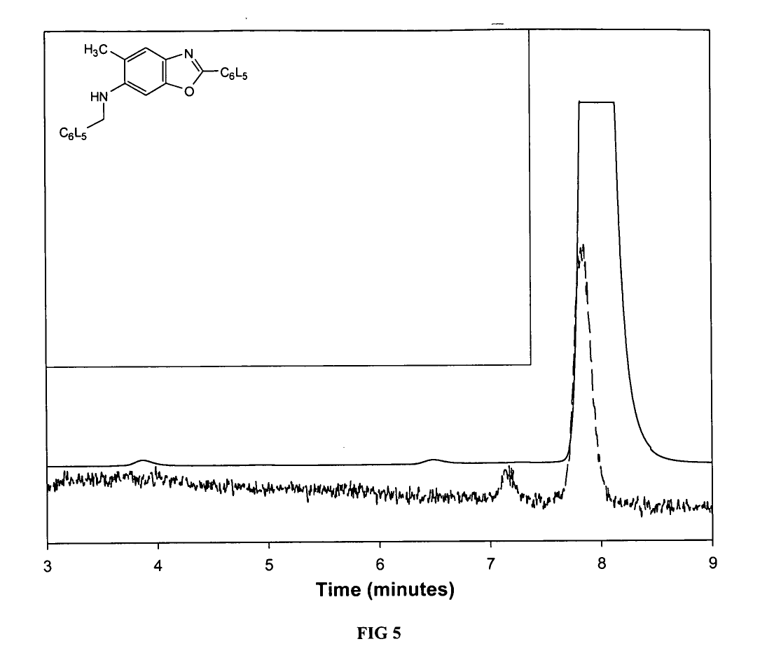

With the derivatization protocol well defined, efforts were directed towards relative quantitation experiments for the model catechol 4-methylcatechol. Two separate 4-methylcatechol solutions (reaction conducted at the 100 nM level) were derivatized with ¹H₅—BA and ²H₅—BA to form “light” (L) and “heavy” (H) versions of the product. When equal volumes of these solutions were mixed and subsequently determined by HPLC-FL-MS, the data shown in FIG. 5 were obtained. The upper chromatographic trace shows fluorescence detection of the co-eluting L and H products, while the lower trace shows the total ion current chromatogram obtained from MS detection. The inset MS data shows the expected product molecular weights and the +10 mass shift resulting from the use of ²H₅—BA. In further experiments, the L and H solutions were prepared in triplicate in various ratios (L/H: 1:6, 2:5, 3:4, 4:3, 5:2, 6:1) and again determined by LC-MS. When the resulting data were subjected to linear regression analysis it was possible to relate the ratio of the prepared standards (L/H_{std}) to the observed ratio (L/H_{obs}) on a relative basis using the following equation: L/H_{obs}=0.1475+1.0325 (L/H_{std}); r²=0.9991. The standard error of the slope and intercept were 0.0157 and 0.0430, respectively. Close scrutiny of these data reveal a slight bias in detection sensitivity towards the L product (slope of 1.0325 instead of unity); however, this was a relatively small effect and in this preliminary experiment this result could potentially be related to a dilution error in the preparation of the two derivatization reactions.

### EXAMPLE 9

### Tagging 2-Aminocresol with Benzylamine

In this example, 2-aminocresol was used as a 3-nitrotyrosine model to verify the reaction with benzylamine (“BA”) formed the same product as with 4-methylcatechol. The reaction was carried out in the same fashion as previously described for 4-methylcatechol.

More specifically, the benzylamine tagging reaction was carried out in 90:10 methanol/water. Stock solutions of 1 μm 2-aminocresol, 100 mM K₃CN₆Fe, and 1 M BA (H5, D5, D7, and N15) were made up in water. Quantities of 100 μl of 2-aminocresol, 100 μl of K₃CN₆Fe, and 100 μl of benzylamine were added to 700 μl of the methanolic solution were stirred and allowed to stand at a constant temperature of 40° C. The reaction was found to be complete within twenty minutes as confirmed by HPLC-FL analysis.

While in this case the chromatographically purified product was more difficult to crystallize, a limited amount of crystals were obtained and were sufficient to conduct the various NMR, liquid chromatographic and MS experiments, which indicated the same product, N-benzyl-5-methyl-2-phenylbenzo[d]oxazol-6-amine, was obtained from 2-aminocresol as previously found from 4-methylcatechol.

LC-FL-MS analysis showed co-eluting peaks for the 2-aminocresol and 4-methylcatechol products on an ODS C18 column running 90% ACN/HOH both containing 0.1% formic acid. Both the 4-methylcatechol and 2-aminocresol products exhibited the same excitation emission profile of 360 nm and 460 nm respectively. LC-MS analysis for both 2-aminocresol and 4-methylcatechol products showed a MH+ of 315 with H5—BA, a MH+ of 325 with D5—BA, a MH+ of 327 with D7—BA, and a MH+ of 317 with N15—BA. The 2-aminocresol BA reaction was scaled up and the product was isolated and analyzed by NMR. The ¹H NMR of the 2-aminocresol and 4-methylcatechol products matched exactly in regards to both shift and integration. The data provides conclusive proof that the identical product is formed with the reaction of either 2-aminocresol or 4-methylcatechol with benzylamine.

### EXAMPLE 10

### Tagging of 5-Hydroxyindole with Benzylamine

In this example, 5-hydroxyindole was used as a hydroxytryptophan model to verify the reaction with benzylamine (“BA”). The reaction was carried out in the same fashion as previously described for 4-methylcatechol.

More specifically, the benzylamine tagging reaction was carried out in 90:10 methanol/water. Stock solutions of 1 μm 5-hydroxyindole, 100 mM K₃CN₆Fe, and 1 M benzylamine were made up in water. Quantities of 100 μl of 5-hydroxyindole, 100 μl of K₃CN₆Fe, and 100 μl of benzylamine were added to 700 μl of the methanolic solution were stirred and allowed to stand at a constant temperature of 40° C.

LC-FL-MS was used to obtain an MH+ of 235 for the product of 5-hydroxyindole and benzylamine. The mass is consistent for incorporation of a single benzylamine forming a benzoxazole ring structure. The 5-hydroxyindole product exhibited a fluorescence excitation emission profile of 375 nm and 475 nm respectively. The orientation of the final structure was determined by COSY NMR analysis of the reaction of 5-hydroxyindole-3-acetic acid (5-hydroxyindoleAA) with benzylamine and was found to be a non-linear ring system.

### EXAMPLE 11

### Tagging of 3,4-Dihydroxyphenylalanine Peptides (DOPA-Peptides) with Benzylamine

In this example, the benzylamine tagging reaction for DOPA-peptides should be carried out in a methanolic solution (90:10 methanol/water). First, stock solutions of 1 μm of DOPA containing peptides, 100 mM K₃CN₆Fe, and 1 M benzylamine (BA) are to be made up in water. Second, quantities of 100 μl of DOPA containing peptides, 100 μl of K₃CN₆Fe, and 100 μl of benzylamine are to be added to 700 μl of methanolic solution. The resulting mixture should be mixed thoroughly and allowed to stand at a constant temperature of 40° C. for 20 minutes.

The resulting sample must be de-salted prior to mass spectrometry analysis. (Example: Solid phase extraction using Waters HLB SPE columns).

It will be appreciated to those skilled in the art that these procedures may be used in conjunction with all of the benzylamine tagging reagents disclosed herein, including Compounds I and II.

### EXAMPLE 12

### Tagging of Hydroxytryptophan Peptides (HT-Peptides) with Benzylamine

In this example, the benzylamine tagging reaction for HT-peptides should be carried out in a methanolic solution. (90:10 methanol/water). First, Stock solutions of 1 μm of HT containing peptides, 100 mM K₃CN₆Fe, and I M benzylamine (BA) are to be made up in water. Second, quantities of 100 μl of HT containing peptides, 100 μl of K₃CN₆Fe, and 100 μl of benzylamine are to be added to 700 μl of methanolic solution. The resulting mixture should be mixed thoroughly and allowed to stand at a constant temperature of 40° C. for 20 minutes.

The resulting sample must be de-salted prior to mass spectrometry analysis. (Example: Solid phase extraction using Waters HLB SPE columns).

It will be appreciated to those skilled in the art that these procedures may be used in conjunction with all of the benzylamine tagging reagents disclosed herein, including Compounds I and II.

### EXAMPLE 13

### Tagging of 3-Nitrotyrosine Peptides (3NT-Peptides) with Benzylamine

In this example, the benzylamine tagging reaction for 3NT-peptides should be carried out in a methanolic solution. (90:10 methanol/water). First, the 3NT-peptides must first be reduced to 3-aminotyrosine peptides (3AT-peptides). Then, stock solutions of 1 μm of 3AT containing peptides, 100 mM K₃CN₆Fe, and 1 μM benzylamine (BA) are to be made up in water. Quantities of 100 μl of 3AT containing peptides, 100 μl of K₃CN₆Fe, and 100 μl of benzylamine are to be added to 700 μl of methanolic solution. The resulting mixture should be mixed thoroughly and allowed to stand at a constant temperature of 40° C. for 20 minutes.

The resulting sample must be de-salted prior to mass spectrometry analysis. (Example: Solid phase extraction using Waters HLB SPE columns).

It will be appreciated to those skilled in the art that these procedures may be used in conjunction with all of the benzylamine tagging reagents disclosed herein, including Compounds I and II.

### EXAMPLE 14

### Generation of DOPA Peptides and Relative Quantitation

This example investigated the generation of two DOPA containing peptides and the relative quantitation using the benzylamine tagging reagents of the present invention. Peptides containing DOPA residues are not readily available from commercial sources, therefore two tyrosine containing peptides VYIHPF (Ang II₃₋₈) and YGG were selected for in situ conversion to the corresponding DOPA-peptides by the action of mushroom tyrosinase.

More specifically, to 5 mg of the tyrosine-containing peptide (Ang II₃₋₈ or Tyr-Gly-Gly) dissolved in 1.0 mL of water was added 5 mg of mushroom tyrosinase and the reaction stirred for 30 minutes at ambient temperature. See Marumo K, Waite J H (1986) Biochem. Biophysica Acta 872:98-103, with modifications allowing for using with the presently developed analytical derivatization reaction.

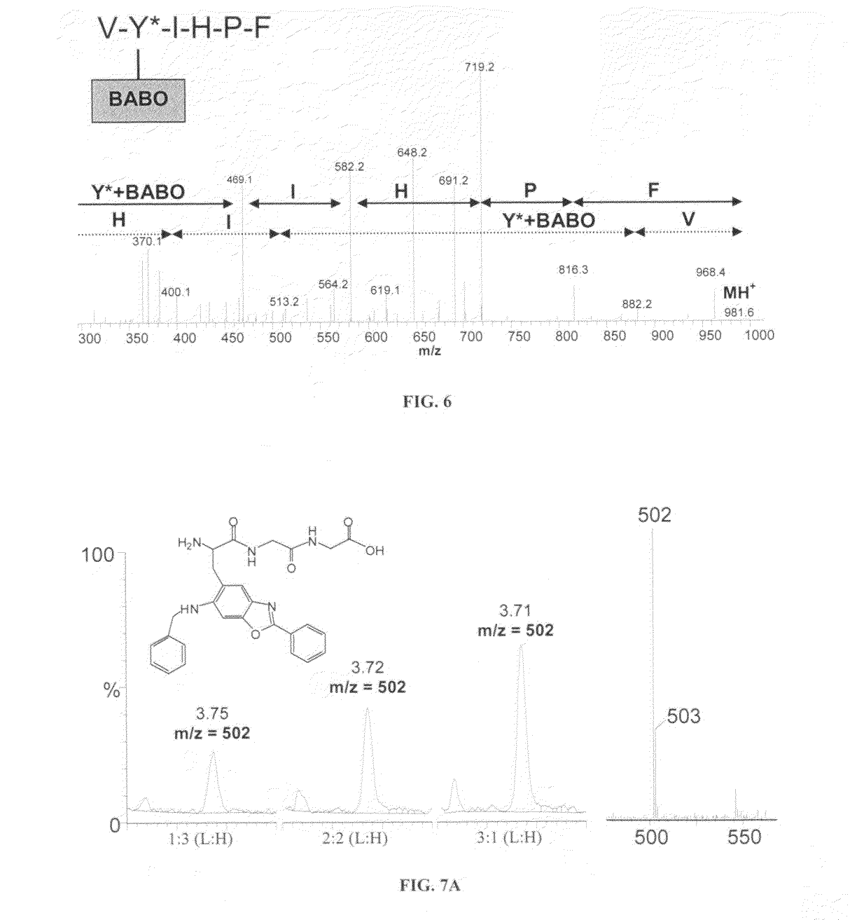

For Ang II₃₋₈ a 100 μL aliquot was derivatized with benzylamine according to the procedure previously described herein. The entire reaction mixture was loaded onto a solid-phase extraction unit (Oasis HLB® cartridges; 1 cc-30 mg; Waters Corporation, Milford, Mass.) unit that had been preactivated by washing with 1 mL of MeOH followed by 1 mL of water. Once loaded, the cartridge was washed with 1 mL of water, with elution accomplished using 2 mL of 8:2 (v/v) ACN:H₂O (0. 1% TFA). The eluent was analyzed by direct infusion ion-trap MS. The product was found to exhibit an MH+ ion with m/z=981.4, indicating the presence of the intact singly charged derivatized BABO peptide. When the 981.4 ion was subjected to an MS-MS experiment, the observed fragmentation pattern was consistent with VY*(BABO)IHPF, the expected product. As shown in FIG. 6, peptide fragments b₂-b₅ and y₅ indicate the presence of the intact derivatized transformed peptide, thus demonstrating the stability of the BABO moiety while the peptide backbone is undergoing collision-induced fragmentation, necessary to obtain sequence information in proteomics investigations.

For Tyr-Gly-Gly, the same protocol for conversion was utilized. To demonstrate relative quantitation, 100 μL aliquots of the resulting DOPA-Gly-Gly were separately derivatized with ¹H₅—BA and ²H₅—BA to form “light” and “heavy” products. Both reaction mixtures were separately purified using solid-phase extraction (cartridges and activation as before). Once loaded, the cartridge was washed with 1 mL of water, with elution accomplished using 2 ml of MeOH and 1 ml of ACN. The samples were evaporated to dryness and reconstituted in 1 ml of 5:5 (v/v) ACN:H₂O (0.1% FA). These two reaction solutions were mixed in various precise ratios (1:3, 2:2, 3:1) to form standards for determination by HPLC-Fl-MS. Relative quantitation was accomplished by peak integration using the MicroMass QuattroMicro™ software.

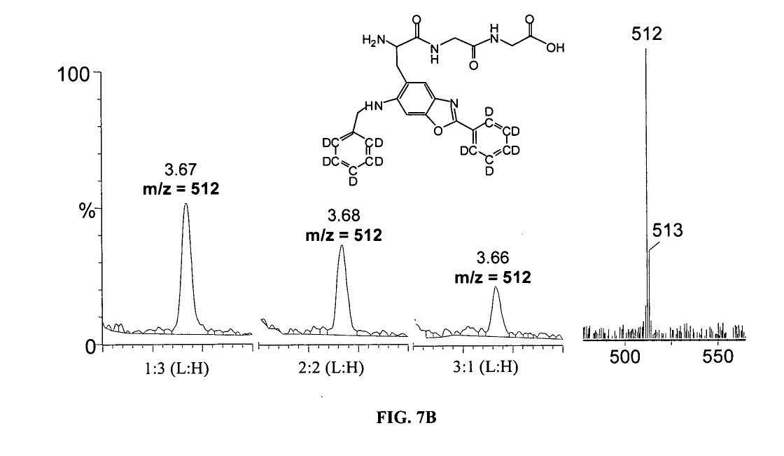

The data obtained by HPLC-MS was analyzed by linear regression and found to be described by the following equation: L/H_{obs}=0.0091+1.1306 (L/H_{std}); r²=0.9999. The standard error of the slope and intercept were 0.0100 and 0.0184, respectively. In FIG. 7A, HPLC-MS chromatograms for the H/L product mixtures (filtered for the “light” product) and the spectrum of the “light” product are shown. In FIG. 7B, similar data are presented for the “heavy” product. While these results are from a limited group of samples, when taken together indicate that no real barriers appears to exist for the derivatization of DOPA peptides with benzylamine. Thus similar to the model 4-methylcatechol, the catechol moiety when present on peptides is appears to be amenable to fluorogenic derivatization with benzylamine and to relative quantitation via the use of L and H isotopes of benzylamine

### EXAMPLE 15

### Tagging of Phosphorylase b with Benzylamine

This procedure is representative for protein tagging, in general, but may be modified slightly to accommodate specific properties of the proteins of interest.

50-μg aliquots of phosphorylase b (PhB) isolated from skeletal muscles of 5 and 36 months old rats (ca. 2 mg/ml protein in the isolation buffer consisting from 20 mM Tris, pH 7.4 , and 100 mM NaCl) were taken and sample volumes were adjusted to 40 μL with the same buffer.

10 μL of 0.2 M sodium dithionite (SDT) in buffer containing 0.5 M Na₂HPO₄ (pH 9.1) were added to obtain pH about 9 and a molar ratio of PhB to SDT of 1:2000. Reduction of protein-bound 3-nitrotyrosine (“3-NT”) to 3-aminotyrosine (“3-AT”) was carried out for 30 minutes at a room temperature (20° C.).

Cleaning the protein samples from unreacted reagents was performed using ProteoSpin™ CBED Micro Kit (Norgen Biotek Corporation, St. Catharines, Ontario, CAN) based on the binding of positively charged protein on ion-exchange column material. The procedure was done in accord with the manufacturer's protocol and included:

Adjustment of pH of protein samples to 4.5 using the pH binding buffer (20 μL).

Column activation by application of 250 μL acidic wash buffer and centrifugation for 1 minute at 14,000×g in a benchtop microcentrifuge (repeated 2 times). The flowthrough discarded.

Protein binding by applying a sample and centrifugation for 1 minute. The flowthrough discarded.

Column wash with 250 μL acidic wash buffer and centrifugation for 1 minute at 14,000×g in a benchtop microcentrifuge (repeated 2 times). The flowthrough discarded.

Protein elution with 25 μL of 100 mM Na₂HPO₄ (pH 9.1) and centrifugation for 1 minute 2 times collecting the flowthrough into the same microcentrifuge tube.

Fluorescent derivatization of protein-bound 3-AT. To 50 μL samples 100 μL methanol, 20 μL 4 mM K₃Fe(CN)₆, and 40 μL benzylamine tag were added and samples incubated for 1 hour at room temperature.

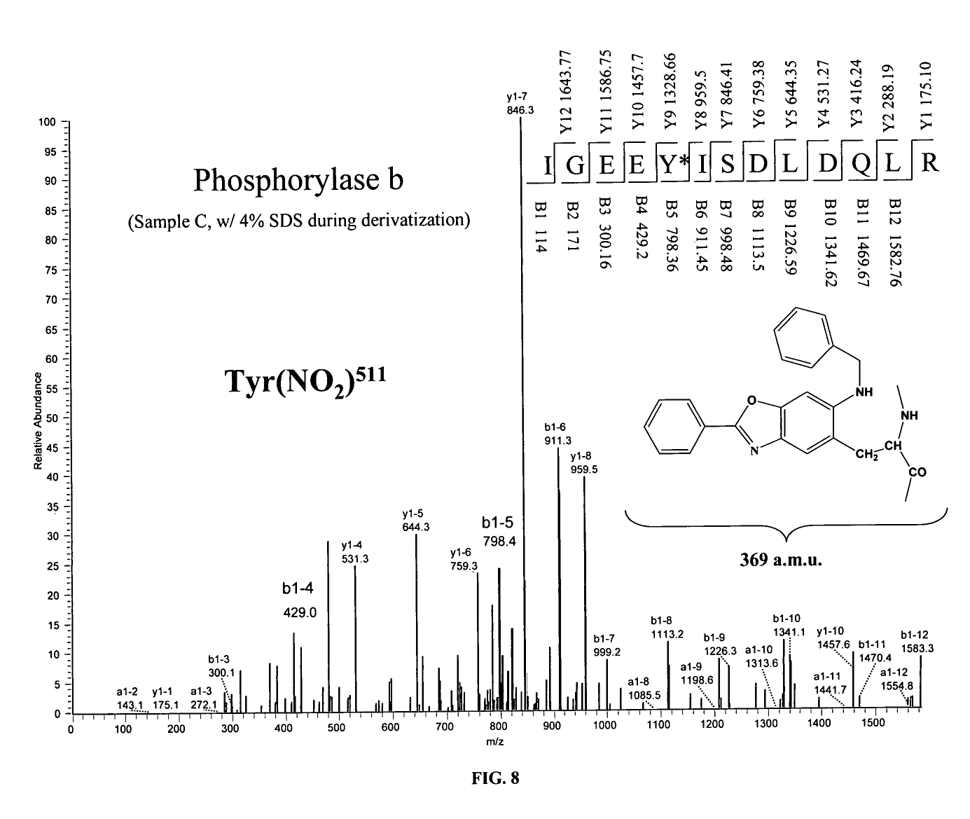

The fluorescence of PhB samples is analyzed either on fluorescence spectrophotometer or by reverse-phase HPLC with fluorescence detection, using excitation and emission wavelengths 360 and 510 nm (for sulfonated benzylamine tag, reagent 1 (4-Sulfobenzylamine)) or 350 and 440 nm (benzylamine), respectively. The results are illustrated in FIG. 8.

Further, the present inventors have also tagged proteins from the homogenate of rat heart with benzylamine (data not shown). Subsequently, size exclusion chromatography was performed to demonstrate that tagging targeted high molecular weight biomolecules (likely proteins. Preliminary mass spectrometry evidence showed tagged sequences in rat heart homogenate.

### EXAMPLE 16

### Tagging of Phosphorylase b with Compound A

In this example, phosphorylase b (a protein of ca. 100,000 Da molecular weight) was successfully tagged with Compound A using 5-200 □M Fe³⁺ and 2 mM compound A. The phosphorylase b protein contained different amounts of 3-nitrotyrosine (see x-axis) as determined independently by UV spectroscopy. The results were analyzed by mass spectrometry. Various amounts of iron were used.

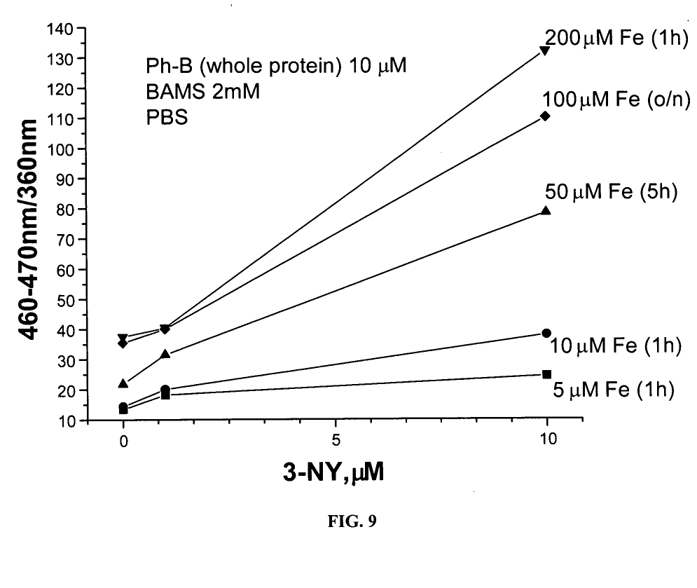

The results are shown in FIG. 9. These results show that while the preliminary tagging procedures required relatively large concentrations of the benzylamine tagging agent (up to 0.5 M) and Fe⁺3 (up to 5 nM), suitable tagging efficiencies may be obtained with concentrations of Fe+3 as low as 10 μM and concentrations of the benzylamine tagging reagent as low as 2 mM.

From the foregoing it will be seen that this invention is one well adapted to attain all ends and objectives herein-above set forth, together with the other advantages which are obvious and which are inherent to the invention. Since many possible embodiments may be made of the invention without departing from the scope thereof, it is to be understood that all matters herein set forth or shown in the accompanying drawings are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. A protein mass tag reagent comprising a benzylamine moiety, wherein said protein mass tag reagent is optionally differentially labeled with one or more isotopic chemical substituents according to: 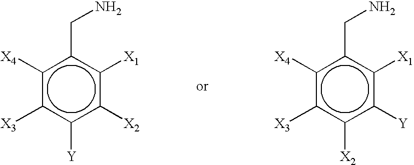
wherein X₁, X₂, X₃, and X₄ are independently H and D; and
wherein Y is H or D, or an accessory moiety.

2. The protein mass tag reagent according to claim 1 wherein said assessory molecule is selected from the group consisting of an enzyme, a radioactive isotope, a heavier isotope, or a fluorophore.

3. The protein mass tag reagent according to claim 1 having following structure (“Compound A”): 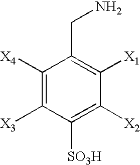
wherein X₁, X₂, X₃, and X₄ are independently H and D.

4. The protein mass tag reagent according to claim 3 wherein X₁, X₂, X₃, and X₄ are all H or all D.

5. The protein mass tag reagent according to claim 1 having following structure (“Compound B”): 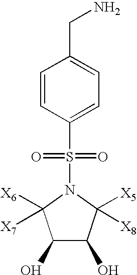
wherein X₅, X₆, X₇, and X₈ are independently H and D.

6. The protein mass tag reagent according to claim 5 wherein X₅, X₆, X₇, and X₈ are all H or all D.

7. A protein tagged with the protein mass tag reagent of claim 1.

8. The protein of claim 7 wherein said protein is tagged at a residue selected from the group consisting of 3,4-dihydroxyphenylanine, 3-nitrotyrosine, and hydroxytryptophan.

9. A method for characterizing a molecule by mass spectrometry comprising: (a) reacting a post-translational modification in said molecule, said modification selected from the group consisting of 3,4-dihydroxyphenylanine, 3-nitrotyrosine, and hydroxytryptophan, with a mass tag reagent having a benzylamine functionality capable of reacting with said post-translational modification; and (b) characterizing the molecule by mass spectrometry.

10. The method according to claim 9, wherein the molecule comprises a protein, a polypeptide, a peptide, or an amino acid.

11. The method according to claim 9 wherein said molecule is attached to a solid support.

12. The method according to claim 9 wherein said mass tag reagent having a benzylamine functionality is defined according to 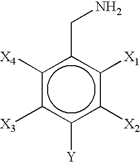
wherein X₁, X₂, X₃, and X₄ are independently H and D; and
wherein Y is H or D, or an accessory moiety.

13. The method of claim 9, wherein said post-translational modification is 3,4-dihydroxyphenlanine in a digested peptide or protein fragment.

14. The method of claim 9, wherein said post-translational modification is 3-nitrotyrosine in a digested peptide or protein fragment.

15. The method of claim 9, wherein said post-translational modification is hydroxytryptophan in a digested peptide or protein fragment.

16. The method of claim 9, wherein said molecule is in a biological sample.

17. A compound according to following structure (“Compound C”): 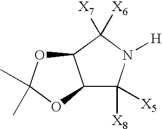
wherein X₅, X₆, X₇, and X₈ are independently H and D.

18. The compound according to claim 17 wherein X₅, X₆, X₇, and X₈ are all D.
